(19) United States
(12) Patent Application Publication
(10) Pub. No.: US 2004/0023304 A1
(43) Pub. Date: Feb. 5, 2004

(54) Biomimetic systems consisting of lipid membranes bound to a substrate

(76) Inventors: Schiller Stefan, Oestrich-Winkel (DE); Naumann Renate, Darmstadt (DE); Knoll Wolfgang, Mainz (DE)

Correspondence Address: SUTHERLAND ASBILL & BRENNAN LLP, 999 PEACHTREE STREET, N.E., ATLANTA, GA, 30309, US

(21) Appl. No.: 10/443,659

(22) Filed: May 22, 2003

(30) Foreign Application Priority Data
May 22, 2002 (DE) 102 22 588.4

(51) Int. Cl. G01N 33/53; C08G 63/91; C08G 63/48

(52) U.S. Cl. 435/7.1; 525/54.1

(57) ABSTRACT

The invention concerns lipids functionalized with a hydrophilic linker which contain a tethering group for binding to a surface. The invention also concerns self-assembled monolayers of lipids on substrates in particular on ultrasmooth gold substrates. The lipids and the lipid monolayers or bilayers containing these lipids can be used to produce biomimetic supported membrane systems. After the optional incorporation of functional molecules such as membrane-associated proteins, these systems can be used for applications such as model systems for examining biological membranes, screening methods, sensors and bioelectronic devices such as biocomputers.

The invention concerns lipids functionalized with a hydrophilic linker which contain a tethering group for binding to a surface. The invention also concerns self-assembled monolayers of lipids on substrates, in particular on ultrasmooth gold substrates. The lipids and the lipid monolayers or bilayers containing these lipids can be used to produce biomimetic supported membrane systems. After the optional incorporation of functional molecules such as membrane-associated proteins, these systems can be used for applications such as model systems for examining biological membranes, screening methods, sensors and bioelectronic devices such as biocomputers.

The rapid progress in the elucidation of biological and medical problems has become impeded by limitations in various fields of analysis which impact not only the field of deciphering the genome of entire organisms (genomics) and the field of proteomics which concerns the verification of the code-function relationships between genome and proteome. Also research on cell-cell interactions and membrane proteins depends to an increasing extent on powerful model systems since they can often only be analysed in a native environment under difficult conditions e.g. using methods such as the patch-clamp technique which are not suitable for wide-scale use (standard analyses and measuring systems). An application of biological model membrane systems which does not necessarily primarily depend on their strict electrical properties but rather on their fluidity and chemical properties is the investigation of the processes described above with regard to their influence on cell-cell interactions and neuro(degenerative) cell changes which are also manifested as modified glycolipid structures on the cell surface (Kolter, Sandhoff, Angew. Chem. Int. Ed. 1999, 38, 1532-1568) or are indicated by the occurrence of changed glycopeptide structures (e.g. in the case of mucins which play an important role in cell interactions (Schiller, S., “Diplomarbeit”, Mainz, 1998)).

The search for biomimetic systems which enable the investigation of such systems or their use in analytics makes high demands on the model system that is used. A basic requirement is the greatest possible agreement between the natural prototype and the biomimetic model which has to be able to imitate the natural prototype with regard to the properties that are important for the problem in question.

One of the most important biomimetic systems consists of a lipid bilayer similar to the biological membrane which is applied to an electrically conductive planar substrate. Such a system enables the investigation of membrane-associated biomolecules (e.g. H⁺-ATPase, cytochrome oxidase, ion channels etc.) which are associated with electrical processes or are controlled by them and are therefore used in biosensor systems and in drug screening.

A large number of applications, their biophysical properties and the diverse chemical compositions with regard to the parts of the molecule that support the membrane and variations in the lipid part (from saturated to unsaturated alkyl chains of different chain length in a monomeric, dimeric or multimeric form which can additionally be provided with hydrophobic side groups and can contain various polar head groups which bind them) and their binding to the aforementioned tethering group which is usually polar are described in Ringsdorf et al., “Angew. Chem. Int. Ed.”, 1988, 27, 113-158; Lang, et al., Langmuir, 1994, 10, 197; Sackmann, Science, 1996, 271, 43-48; Guidelli et al., J. Electroanal. Chem., 2001, 504, 1-28 and Knoll et al., Mol. Biotechnol., 2000, 74, 137-158.

The functional binding of the lipid membrane to a solid substrate via a flexible polar (hydrophilic) intermediate part is an approach that has been popular for years (Sackmann, see above) and has the following advantages: Firstly it achieves a robust binding of the sensitive lipid bilayer to the solid substrate which, due to its planarity, enables a number of sensitive electrical and surface-analytical methods to be used and also uncouples the membrane from the surface. The uncoupling from the surface suppresses the hydrophobic interaction which occurs on metal surfaces and which is negative for the membrane and increases the cytosol-like volume between the membrane and substrate surface which is of fundamental importance especially for volume-dependent transport processes as well as for generating fluid membranes.

The measuring methods that can be used for membrane systems comprise in the case of electrical methods for example impedance spectroscopy (IS), voltammetry and other potentiometric and amperometric methods such as chrono-coulometry. Methods for analysing surfaces comprise a number of methods, many of which require planar metal films as a proximal layer before modification with the system to be analysed. They often allow an analysis down to submolecular orders of magnitude which can only be achieved by such solid body supported membranes. These include for example surface plasmon resonance spectroscopy (OPR, SPR or SPS) for determining the layer thickness (refractive index) of compounds applied to the substrate (e.g. Au or Ag films). However, a large number of other measuring methods some of which are very sensitive can be used to characterize the described system. These include methods such as ellipsometry, fluorescence methods, internal reflectometry, light scattering methods, X-ray or neutron scattering, surface acoustic methods, utilization of thermal effects (changes in the temperature or heat flow), measurement of masses or changes in density which can for example be determined with a quartz microbalance, measurement of changes in the membrane phase, radioimmunoassays or enzyme-linked assays.

Realization of a model system which allows the application of the said methods and, on the other hand, meets the requirements for a functional biological model membrane having the necessary properties is a problem which makes very high demands on the molecular properties of the membrane components, their surface binding, the molecules which surround them, the properties and quality of the surface and the measuring methods that are to be used.

Numerous examples of supported membrane systems are known from the prior art. For example membrane systems based on thiol-modified oligopeptides and thiol lipopeptides can be used (see e.g. Bunjes et al., Langmuir, 1997, 13, 6188-6194; Naumann et al., Bioelectrochem. Bioenerg. 1997, 42, 241-247; Schmidt et al., Biosensors. Bioelectron. 1998, 13, 585-591; Naumann et al., Biosens. Bioelectron. 1999, 14, 651-662 and WO 96/18645 and WO 99/20649). Peggion et al., Langmuir, 2001, 17, 6585-6592 also describe oligopeptides as membrane-supporting components that are provided with polar triethylene glycol side chains and form a hydrophilic monolayer.

Membrane systems hydrophilically supported by polyethylene glycol are described by Lang et al., Langmuir, 1994, 10, 197-210, Heyse et al., Biochem.Biophys. Acta 1998, 85507, 319-338, EP-A-0 441 120 and EP-A-0 637 384. Another membrane system supported by a short oligoethylene glycol chain which has cholesterol as a hydrophobic domain is described by Williams et al., Langmuir 1997, 13, 751-757 and Jenkins et al., Langmuir, 1998, 14, 4657.

Other membrane systems are described by Cornell et al., Nature 1997, 387, 580-583, Raguse et al., Langmuir 1998, 14, 648-659, Krishna et al., Langmuir 2001, 17, 4858-4866 and in WO 89/01159, WO 90/02327, WO 94/07593, WO 97/01092, U.S. Pat. No. 5,753,093 and U.S. Pat. No. 5,783,054. The components used to build-up the membrane are composed of various monophytanyl/monophytanoyl-oligoethylene glycol thiols/disulfides, polar lateral spacer molecules and a membrane-spanning thiolipid. Functionalization molecules such as gramicidin can be incorporated into the membrane. After treating a gold surface with an ethanolic solution of the individual components, the membrane is generated in situ by rinsing with buffer.

Disadvantages of the above-mentioned membrane systems are that often inadequate electrical properties are obtained especially with regard to the capacity and resistance when they are bound to an electrically conductive substrate surface. Moreover, some of the membrane systems known from the prior art have an inadequate chemical stability. Hence one of the objects of the present invention was to provide new membrane systems which at least partially eliminate the above-mentioned disadvantages. In particular the membrane systems according to the invention should have improved electrical properties compared to the prior art.

This object is achieved by providing new tethering lipids which are suitable for anchoring membranes to a substrate surface in particular an electrically conductive substrate surface. These tethering lipids contain a carrier comprising at least two functionalities and preferably three or four functionalities. The carrier can for example be an organic group having 3-10 C atoms which contains hydroxy, thiol or/and amino groups as functionalities. Preferred functionalities are hydroxy groups. Other residues that are defined in the following can be bound to these functionalities e.g. by means of ether bonds, thioether bonds, ester bonds or/and amide bonds. Preferred examples of carriers are glycerol, 1-amino-propane-2,3-diol, 2-amino-2-hydroxymethyl-1,3-propanediol (Tris), amino acids such as serine, threonine, lysine etc., polar compounds with thiol functionalities such as dimercaptosuccinic acid, sugar alcohols such as mesoerythritol, threitol etc., sphingosins and similar glycerol derivatives, tri to pentaamines etc. Glycerol is a particularly preferred carrier.

At least one saturated or unsaturated hydrocarbon or acyl residue having a chain length of 10-22 C atoms which can optionally be substituted by one or more side groups e.g. C₁-C₄ alkyl groups and in particular methyl groups is bound to the functionalities of the support. At least two such hydrocarbon or acyl residues, e.g. 2 or 3 of them, are preferably bound to the carrier.

In addition a residue comprising an oligoethylene oxide group in particular a group (CH₂CH₂O)ₙ in which n=1-100 and a tethering group are bound to the carrier. Preferably 2-80, particularly preferably 3-70 and most preferably 4-50 ethylene oxide units are present.

The tethering or anchoring group which may be used for anchoring to a substrate is preferably a group which enables an adsorptive or covalent binding to a metallic or oxidic surface. Preferred examples are groups containing sulfur such as thiol or disulfide groups which are particularly suitable for anchoring to metallic surfaces.

However, non-sulfurous groups can also be used such as phosphine groups or CN groups (Xia and Whitesides, “Angew. Chem.” Int. Ed. 1998, 37, 550-575). S—S (disulfide) groups are particularly preferably used as tethering groups and cyclic S—S groups are most preferably used. A preferred example of the group R³ is a lipoic acid residue.

In addition other groups can be optionally bound to the carrier such as short-chained e.g. C₁-C₉ hydrocarbon residues or/and residues comprising phospholipid, carboxyl, carbonyl, SO, SO₂, amide, amino groups with and without C₁-C₉ hydrocarbon residues, e.g. tetrazole, thiol groups (and the aforementioned groups).

The tethering lipids are particularly preferably compounds of the general formulae (Ia) and (Ib):

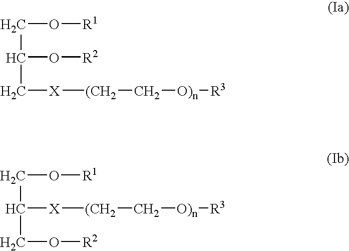

in which at least one of R¹ and R² is independently a saturated or unsaturated hydrocarbon residue or an acyl residue having a chain length of 10-22 C atoms which can optionally be substituted by one or more side groups e.g. C₁-C₄ alkyl groups and in particular methyl groups, and the other can be hydrogen, a C₁-C₉ hydrocarbon residue or a residue comprising a phospholipid, carboxyl, carbonyl, SO, SO₂, amide, amino or thiol group with or without a C₁-C₉ hydrocarbon residue,

n is an integer from 1-100,

R³ is a residue containing a tethering group and

X is a linking group.

In this connection the compounds (Ia) are 1,2 sn as well as 2,3 sn lipids according to the relevant stereochemical nomenclature for lipids (sn).

At least one of R¹ and R² is preferably a saturated or unsaturated hydrocarbon or acyl residue which is substituted by one or more methyl groups. R¹ and R² are particularly preferably such hydrocarbon or acyl residues. The chain length of R¹ and R² is preferably 12-20 C atoms and particularly preferably 13-18 C atoms. Examples of suitable hydrocarbon and acyl residues are phytanyl, phytanoyl, lauryl, lauroyl, tridecanyl, tridecanoyl, myristyl, myristoyl, pentadecanyl, pentadecanoyl, palmityl, palmitoyl, oleyl, oleoyl, linoleyl, linoleoyl, arachidonoyl, docosahexaenyl, docosahexacnoyl etc. Hydrocarbon residues are particularly preferred which are bound to the support via an ether group such as phytanyl.

The linking group X is preferably selected from O, S or NR in which R represents H or a C₁-C₄ alkyl residue. X particularly preferably represents O.

The compounds of the general formulae (Ia) and (Ib) are preferably glycerol derivatives containing in particular 2 hydrocarbon or/and acyl residues and a residue for anchoring to a substrate which residue contains an oligoethylene oxide group having for example 1-100 ethylene oxide units.

In a particularly preferred embodiment the compound (I) is selected from 2,3-di-O-phytanyl-sn-glycerol-1-tetraethylene glycol-DL-α-lipoic acid ester and optical isomers thereof. Other examples of suitable compounds are diphytanyl/oyl-phosphatidyl-tetraethylene glycol lipoic acid esters, corresponding compounds in which the lipoic acid structure carries an ether or carbon-carbon linkage instead of the ester linkage and the stereochemistry at the glycerol component also contains the corresponding 1,2- and 1,3-di-O-phytanyl linkages. It is also possible to select another carrier for the phytanyl groups instead of the glycerol components such as Tris[2-amino-2-hydroxymethyl-1,3-propane diol] which carries an additional polar hydroxyl group or which is able to receive a third phytanyl group.

The compounds (I) according to the invention are characterized in that they contain two unpolar lipid groups which are bound to a carrier component, e.g. a D-glycerol component, via an ether or ester bond. The support component contains an oligoethylene glycol chain on its third functionality, e.g. on a hydroxy group which is preferably also bound via an ether group. The stricture of the compounds according to the invention make them less sensitive to hydrolysis which is very advantageous for long-term applications. A disulfide group is preferably used as a tethering group and a cyclic disulfide group is particularly preferred. Both sulfur atoms of the disulfide are preferably covalently linked with the polar oligoethylene oxide residue.

The compounds (I) can be anchored to a substrate surface by means of the tethering group. This anchoring is preferably an adsorptive binding which ensures that individual molecules of compounds (I) are still able to move laterally on the substrate surface. In addition to the molecules of compounds (I), spacer molecules can also be bound to the substrate surface i.e. molecules having a tethering group and optionally a hydrophilic group in order to dilute the molecules of compounds (I). Examples of suitable spacer molecules containing sulfurous tethering groups are lipoic acid, thioethanol, thiodiglycolic acid, cysteine, thioacetic acid, dimercaptosuccinic acid, hydroxy/carboxyalkylthiols and disulfides with a chain length of 1-12 carbon atoms and analogous dialkylsulfides (thioethers) and also hydrophobic compounds such as alkylthiols and disulfides having a chain length of 1-12 carbon atoms and analogous thioethcrs, phenylthiols, bisphenylthiol and others. The molar ratio of molecules of compounds (I) to spacer molecules is preferably in a range of 100:0.05 to 0.05:100, particularly preferably in a range of 0.1:100 to 100:1 and most preferably of 15:100 to 70:1. Of course pure tethering lipid layers can also be used.

Another aspect of the invention is a supported biomimetic membrane comprising a lipid layer containing at least one compound of the general formula (I) as tethering lipid for anchoring to a substrate. The lipid layer may comprise a monolayer or, if additional mobile lipids are present, a bilayer.

The substrate preferably has an electrically conductive surface, for example a metal surface and in particular a noble metal surface such as gold, silver or platinum. A gold surface is particularly preferred.

Furthermore a substrate is particularly preferred which has an ultrasmooth surface with a surface roughness of or below the magnitude of a molecule dimension e.g. a maximum of 2-3 nm, preferably below 2 nm, particularly preferably below 1 nm and 0.5 nm in the example shown. An example of such an ultrasmooth surface is an ultrasmooth gold surface which can be prepared by template moulding gold surfaces (Wagner et al., Langmuir, 1995, 11, 3867-3875). An ultrasmooth surface provides an optimal supramolecular arrangement of the individual functional sections of the compounds (I) allowing a particularly advantageous hydrophobic interaction between the hydrocarbon chains.

A supported lipid bilayer can be formed from a monolayer containing compounds of the general formula (I) by adding mobile lipids for example by vesicle fusion. Examples of suitable mobile lipids are phospholipids such as 1,2-di-O-phytanyl-sn-glycero-3-phosphocholine (DphyPC), 1,2-di-O-phytanoyl-sn-glycerophosphocholine, 1,2-di-O-phytanyl-sn-glycerophosphoethanolamine, 1,2-di-O-phytanoyl-sn-glycero-phosphoethanolamine, corresponding 1,2- or 2,3-diphytanyl or diphytanoyl derivatives and mixtures of several of the said compounds or other phospholipids such as egg phosphatidylcholine, DMPC (dimyristoylphosphatidylcholine), DMPE (dimyristoylphosphatidylethanolamine), lecithins and non-phosphorous-containing lipids in a saturated and unsaturated form, provided with identically or differently substituted or non-substituted alkyl chains having preferably 8-22 carbon atoms, in particular with two phytanyl or/and phytanoyl groups which are bound to glycerol by means of ether or/and ester bonds, glycolipids, quartemary ammonium compounds having two C₁-C₄ alkyl groups and two C₈-C₁₈ alkyl chains, tertiary amines having two C₈-C₁₈ alkyl chains and one C₁-C₄ alkyl chain or a hydrogen atom instead of the C₁-C₄ alkyl chain, steroids or chemically similar compounds which can be derived from the cholestane structure or isoprenoids as well as lipid mixtures consisting of the said compounds where the lipids can optionally contain polar head groups such as hydroxyl, carboxyl, phosphoric acid ester and derivatives thereof, sulfur oxide derivatives having various oxidation stages of the sulfur and naturally occurring groups such as cholines, ethanolamines, inositols, glycerols, aminoglycerols (in sphingosines) and amino acids and neutral, cationic or anionic forms or derivatives thereof. If vesicles are used with membranes in which functional molecules such as membrane proteins are integrated, these can be directly incorporated into the supported membrane during the vesicle fusion.

The lipid bilayer can also be prepared by LB (Langmuir-Blodgett) transfer of lipid layers, spontaneous bilayer formation, by dilution of for example an ethanolic lipid solution with water or buffer solution or by reverse osmosis and dilution of vesicular solutions.

Another advantage of the supported membrane according to the invention is its simple structure since it can be composed of a monolayer with only a single compound (I). It is not necessary to use membrane-spanning lipids which are complicated to synthesize and hence expensive. Furthermore, a substrate such as a gold carrier modified with a monolayer can be stored in a dry state without loss of quality and be simply converted into a functional biological model membrane as required by vesicle function or another of the methods described above. In contrast special measures are needed for the storage of systems of the prior art since in this case only complete model membranes can be prepared and stored which would immediately disintegrate on drying without the addition of special compounds. In addition a considerable loss of quality can occur with such systems even after a short storage.

The supported membranes according to the invention have excellent electrical properties. Hence they can have a capacitance of 0.4 to 0.7 μF/cm², preferably of 0.4 to 0.6 μF/cm². The resistance is preferably at least 1 MΩ·cm², particularly preferably at least 5 MΩ·cm², for example in the range of 6-24 MΩ·cm².

Functional molecules can be incorporated into the lipid membrane such as proteins, e.g. membrane-associated proteins, such as H⁺ ATPases, cytochrome oxidases, ion channels etc. which are associated with electrical processes, other membrane proteins, light-dependent proteins such as bacteriorhodopsin, antibiotics, membrane receptors and ligands e.g. peptides or proteins or other structures containing carbohydrate or/and lipid, or structures composed of heterocycles or combinations thereof.

The supported membranes according to the invention can be used for a large number of applications for example in biosensors and to screen for active substances e.g. to identify or/and characterize pharmacologically active substances. Furthermore the supported membranes can be used to analyse biomolecules such as membrane-associated biomolecules selected from proteins, antibiotics etc. In addition the membranes can be used for electrotechnical applications in particular as electrical insulating materials due to their high resistance and their effective charge separation. They can be used for example in bioelectromechanical micro and nano devices (Bio-MEMS/NEMS) to electrically insulate or embed macroscopic or/and molecular electrical components and for molecular wires (Aviram and Rathner, Chem. Phys. Let. 1974, 29, 257), especially because they have a thickness in a molecular order of magnitude. Moreover, due to their compartmentation capability, the membranes are also able to serve as systems which can for example provide the system components for an interface in biomechanical applications in the field of ATP-dependent electric motors such as e.g. myosin, kinesin and dynein (Taylor et al., Nanotechnology, 1999, 10, 237-243) especially when they are combined with ATP-generating membrane systems or other functional systems e.g. light-dependent proteins such as bacteriorhodopsin.

The invention is further elucidated by the following figures and examples.

FIG. 1: 2,3-di-O-phytanyl-sn-glycero-1-tetraethylene glycol-DL-α-lipoic acid ester (DPTL)

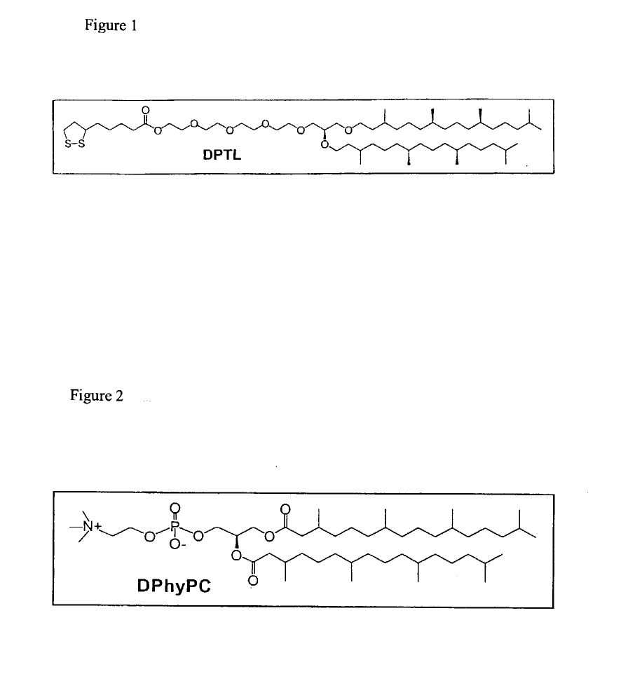

FIG. 2: 1,2-di-O-phytanoyl-sn-glycero-phosphocholine (DPhyPC)

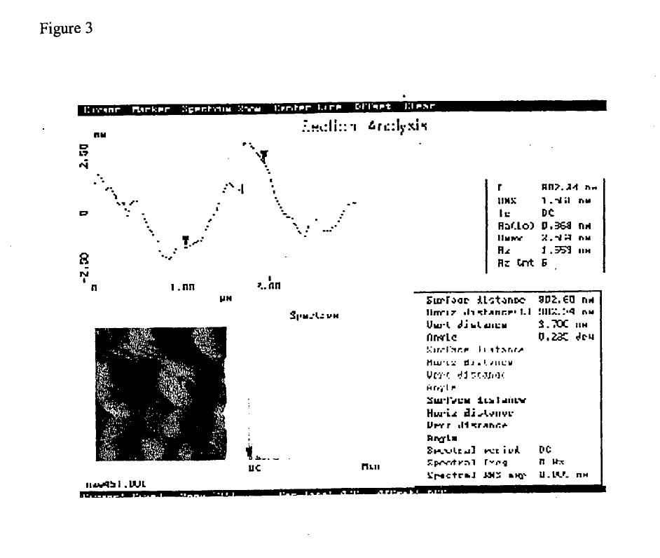

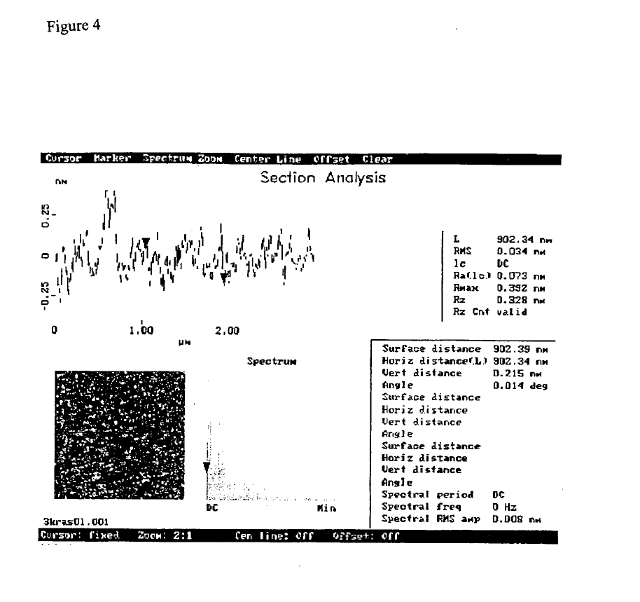

FIGS. 3 and 4: Scanning force micrographs of gold substrates

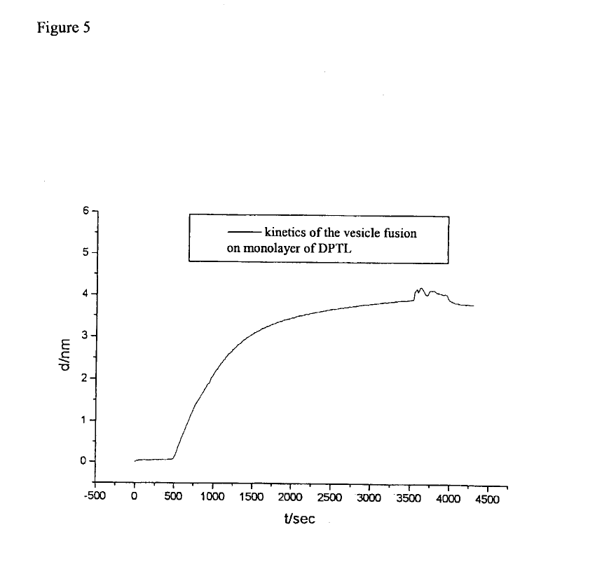

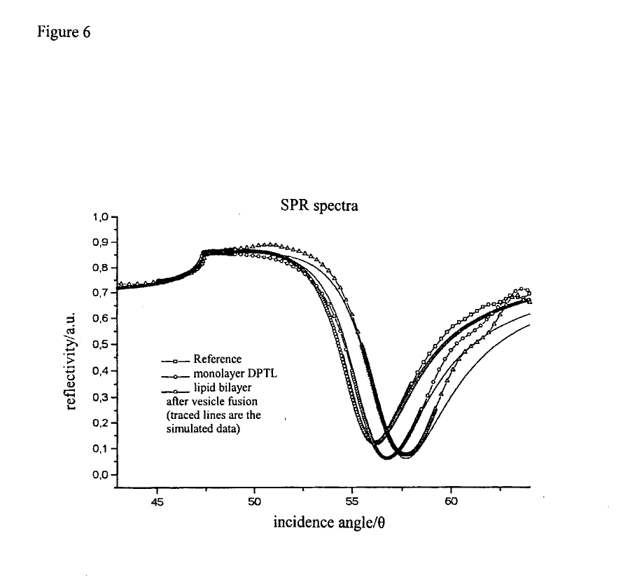

FIG. 5: Kinetics of a vesicle fusion with a DPTL monolayer

FIG. 6: Surface plasmon resonance spectrum (SPR) spectrum of a DPTL layer before and after vesicle fusion

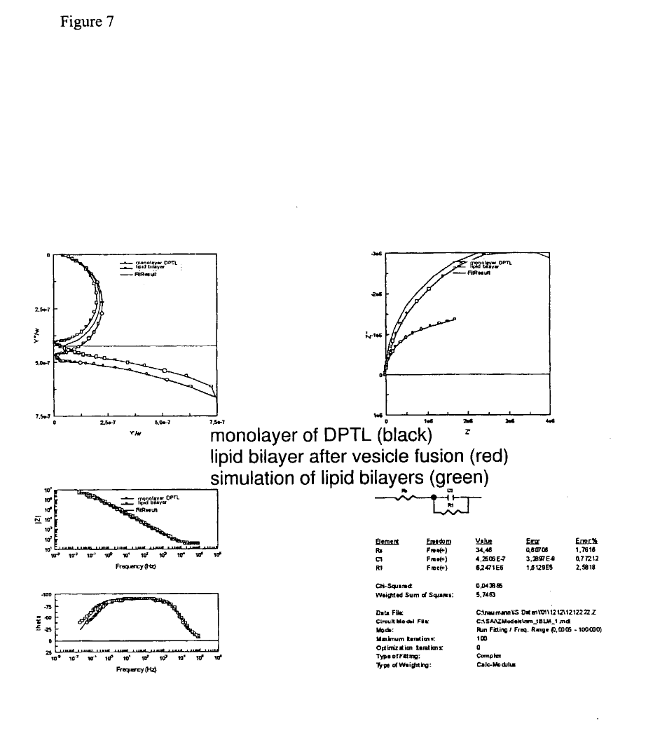

FIG. 7: Impedance(IS) spectrum of a DTPL layer before and after vesicle fusion

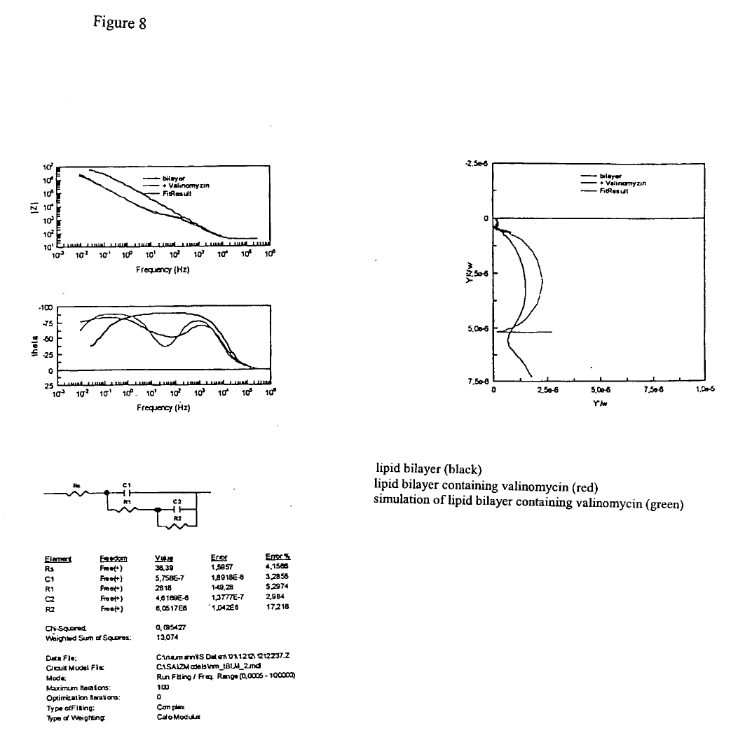

FIG. 8: Spectra of a DTPL lipid bilayer before and after incorporation of valinomycin

### EXAMPLES

### Example 1

Synthesis of 2,3-di-O-phytanyl-sn-glycero-1-tetraethylene-glycol-DL-α-lipoic acid ester

Compounds according to formula (I) e.g. 2,3-di-O-phytanyl-sn-glycero-1-tetraethylene-glycol-DL-α-lipoic acid ester and their starting materials can be prepared by known methods such as those that are described in the literature (e.g. textbooks such as Houben Weyl, “Methoden der organischen Chemie”, Georg Thieme Editors, Stuttgart) under reaction conditions that are used for the aforementioned variants and those variants that have not been elucidated in more detail.

D-mannitol is reacted under standard conditions as described by Baer, J. Amer. Chem. Soc. 1945, 67, 338, to form the diisopropylidene-protected compound (1,2; 5,6-diisopropylidene-D-mannitol). This is cleaved oxidatively by sodium periodate at 0° C. in water to form the aldehyde and the solution is processed further after complete reaction with ethanol, the resulting precipitate is separated and the remaining solution is admixed in an ice-bath with sodium borohydride suspended in water and the aldehyde is reduced to 2,3-isopropylidene-D-glycerol. The reaction mixture is stirred overnight and on the next morning it is adjusted to pH 7 with acetic acid. Subsequently the organic solvent is removed by distillation. The aqueous phase is extracted with methylene chloride and the organic phase is dried with sodium sulfate/sodium carbonate. After removing the solvent, the isopropylidene-protected glycerol component is obtained. The isopropylidene-protected glycerol component is taken up in chloroform and dried with a molecular sieve (3 Å).

All further steps until processing are carried out under an argon atmosphere. P-toluenesulfonyl chloride is added to the solution and pyridine (dried over potassium hydroxide) is added dropwise after 15 min. The ice-bath is removed and the mixture is stirred overnight. The mixture is extracted with water and saturated sodium chloride solution and the organic phase is dried over sodium sulfate and the solvent is removed by distillation. The resulting crude product is purified by flash chromatography on silica gel using petroleum ether/ethyl acetate mixtures as the mobile phase.

Tetraethylene glycol is dissolved in tetrahydrofuran (dried over calcium hydride) and dried over a molecular sieve (3 Å) (the flask is filled with argon). The molecular sieve is removed and the tetraethylene glycol is deprotonated with a mole equivalent of sodium hydride. Afterwards the tosylated, isopropylidene-protected glycerol component is added dropwise, dimethylaminopyridine is subsequently added and firstly stirred for 6 h at room temperature and subsequently for 3 days at 60° C. under an argon atmosphere. The mixture is filtered over a Celite® filter gel, the solvent is removed by distillation and the crude product is diluted with dichloromethane and extracted with water. The organic phase is dried with sodium sulfate and the solvent is removed by distillation. The resulting product phase is taken up in dry dimethyl-formamide (distilled over calcium hydride), cooled to −2.5° C. and deprotonated with sodium hydride. Afterwards benzyl bromide is added dropwise at 0° C. while the temperature is kept below 10° C. After completion of the addition, the cold bath is removed and the mixture is stirred for two days at room temperature under an argon atmosphere. The mixture is filtered over a Celite® filter gel, the solvent is removed by distillation. The crude product is taken up in dichloromethane, extracted with water and purified by flash chromatography on silica gel with a mobile dichloro-methane phase. The benzyl-tetraethylene glycol-2,3-isopropylidene-D-glycerol component obtained is admixed with tetrahydrofuran/water and the isopropylidene protecting group is cleaved for 36 h at 40° C. with trifluoroacetic acid. The reaction mixture is neutralized with sodium carbonate and extracted with dichloromethane after further addition of water. The organic phase is dried, the solvent is removed by distillation and the crude product obtained is purified by flash chromatography on silica gel with a mobile phase of ethyl acetate and acetone with an increasing acetone gradient.

For the modification of the product with two phytanyl residues, phytol is firstly hydrogenated for 2 days to phytanol with platinum oxide in methanol at 40° C. and 70 bar hydrogen pressure. The resulting crude product is then purified by gel filtration on silica gel with hexane. The phytanol obtained is dissolved in dichloromethane and bromated overnight at a temperature of less than 30° C. using triphenyl-phosphine and N-bromosuccinimide. The dichloromethane is removed from the crude product by distillation, the residue is extracted with hexane and filtered over a glass frit. The hexane is removed by distillation and the crude product containing phytane bromide is purified with hexane on silica gel by flash chromatography.

The D-glycerol component modified with tetraethylene glycol protected on one side with benzyl ether in which the 2 and 3 position were deprotected by cleaving the isopropylidene residue with trifluoroacetic acid, is dissolved in dry dimethyl-formamide, deprotonated with sodium hydride and phytane bromide is added under an argon atmosphere using a dropping funnel. The flask is stirred for 4 days at room temperature with exclusion of light. After the addition of water it is extracted with dichloromethane, the organic phase is dried with sodium sulfate and the solvent is removed by distillation. The crude product is purified by flash chromatography on silica gel using a mobile petroleum ether/ethyl acetate phase. The 2,3-di-O-phytanyl-sn-glycero-1-tetraethylene glycol-O-benzyl ether is dissolved in tetrahydro-furan/methanol and the benzyl ether protecting group is cleaved hydrogenolytically with palladium catalysis at room temperature analogous to standard methods. After 2 h the solvent is removed by distillation and the mixture is purified by flash chromatography on silica gel using petroleum ether/acetone as the mobile phase.

The product obtained is dissolved in dry dichloromethane to which DL lipoic acid containing N-(3-dimethylaminopropyl)-N′-ethyl-carbodiimide hydrochloride had previously been added and stirred for 20 min. After 30 min dimethylaminopyridine is added to this solution and the reaction is stirred for 4½ h. The mixture is filtered over Celite® filter gel, the crude product is diluted with dichloromethane and extracted with saturated sodium hydrogen carbonate solution and water. The organic phase is dried with sodium sulfate and the solvent is removed by distillation. It is purified by flash chromatography on silica gel using petroleum ether/acetone as the mobile phase. The purity of the products and their identity is confirmed by thin layer chromatography, NMR and IR spectroscopy, polarimetry and elemental analyses.

### Example 2

Preparation of Gold Substrates (Gold Detached from Mica)

Mica is cut into thin layers under extremely clean conditions such that the upper layer is composed as far as possible of a closed crystalline surface. In order to remove any adhering water, the mica platelets are heated in a tube furnace at 650±1° C. in a gentle flow of nitrogen during which they are moved on a steel-wire holder to the middle of the furnace tube within 15 s, they are kept in the middle for 30 s and removed from the furnace within a further 15 s. Afterwards they are placed in an electrothermal vaporization apparatus (e.g. electrothermal evaporator, Edvards) and immediately evacuated. A gold film with plasmon capability e.g 60 nm is vapour deposited at a pressure of <5×10⁻⁶ mbar and an evaporation rate of <0.1 nm/s. After cooling the mica substrates vapour coated with gold are taken out of the vaporization apparatus and immediately tempered under the same conditions that were used when heating the mica substrates (650° C., flow of nitrogen, 15 s, 30 s 15 s) in the process of which the gold adopts the crystalline structure of Au(111). It was possible to verify this by scanning force microscopy (AFM) (FIG. 3).

Afterwards the gold side of the mica gold substrates is glued onto glass substrates (e.g. high refractive index glass—LaSFN9, if the substrates are also to be examined by SPR) whereby the glue is evenly distributed over the entire area by pressing. After drying at 150° C. (60 min) the substrates are placed for 2-3 min in tetrahydrofuran. Subsequently the mica platelets are removed so that the gold side which previously faced the mica (smooth) now faces upwards. The scanning force micrograph (FIG. 4) now shows a more finely grained but smoother surface, compared with the roughness of Au(111), FIG. 3 and TSG, FIG. 4.

The finished substrates are expediently immediately immersed in a coating solution. Otherwise they can also be stored in pure ethanol.

### Example 3

Preparation of Monomolecular Layers (Self Assembled Monolayers (SAM's)) of DPTL by Self-Organisation on Gold

The gold substrates are stored for 24 hours in a solution of DPTL in ethanol (0.2 mg/ml), afterwards rinsed with ethanol, dried in a flow of nitrogen and stored in an argon atmosphere until use.

### Example 4

Preparation of Tethered Lipid Bilayers by Vesicle Fusion of the Monolayer and Measuring their Layer Thicknesses and Electrical Properties

The substrates coated with a DPTL monolayer are flooded in a buffer solution containing 0.1 mol/l KCl, 0.01 Tricine, 0.01 mol/l Na₂HPO₄, 0.0002 mol/MgSO₄ in water, pH=7.4 in an apparatus for combined surface plasmon resonance spectroscopy (SPR) and impedance spectroscopy (IS) and the appropriate measuring cell see e.g. Bunjes et al. (Langmuir, 1997, 13, 6188-6194). Then the layer thickness is measured by means of SPR, the capacitance and resistance of the monolayer are measured by IS. The temperature of the measuring cell is 32° C. Afterwards 50 μl of a liposome suspension of diphytanoylphosphatidyl choline (DPhyPC) (4 mg/ml of the above-mentioned buffer) freshly prepared by extrusion (through a 50 nm filter) is added to the 1 ml measuring cell. The increase in the layer thickness is monitored by SPR versus time. After achieving a stationary state and rinsing with pure buffer solution, the layer thickness is measured by SPR and the electrical properties are measured by IS.

The results of these measurements are summarized in table 1 in comparison to the known values for the BLM.

TABLE I

| | | Rₘ/MΩ | | | | |
|---|---|---|---|---|---|---|
| | Cₘ/μF cm⁻² | cm² | Cₘ/μF cm⁻² | Rₘ/MΩ cm² | d/nm | d/nm |
| | measured | measured | BLM | BLM | measured | calculated |
| DPTL | 0.5 | 2.5 | | | 4.7 | 4.7 |
| monolayer | | | | | | |
| before vesicle | | | | | | |
| fusion | | | | | | |
| lipid bilayer | 0.42 | 6.2 | 0.5 | 10 | 8.5 | 8 |
| after vesicle | | | | | | |
| fusion | | | | | | |

The kinetics of the vesicle fusion by SPR is shown in FIG. 5 and the SPR and IS spectra before and after vesicle fusion are shown in FIGS. 6 and 7.

A comparison with simulated data (see also FIGS. 6 and 7) show the uniformity of the layers. A comparison with electrical data of pure lipid bilayers (bilayer lipid membranes (BML's)) or with layer thicknesses calculated from molecule dimensions show good agreement. A comparison with data in the literature for lipid bilayers anchored on gold also prepared by vesicle fusion shows the much better agreement of the layers described here made of DPLT with BLM data (membranes that are comparable with biological membranes are stated in the literature to have a membrane resistance of more than 10 MΩ cm² (Sackmann, Wagner, J. Phys. Chem. B, 2002)) particularly with regard to the resistance.

### Example 5

Functionalization of an Anchored Lipid Bilayer with Valinomycin

50 μl of a valinomycin solution (8×10⁻⁵ M in ethanol) is added to the lipid bilayer prepared as described above in a 1 ml measuring cell and in the above-mentioned buffer solution. The IS spectra before and after incorporating valinomycin are shown in FIG. 8 together with the data adapted to the replacement circuit. Capacitance and resistance of the adapted data are summarized in table 2.

TABLE 2

| | Cₘ/μF cm⁻² | Rₘ/MΩ cm² | Cₛₚ/μF cm² | R_{ch}/Ω cm² |
|---|---|---|---|---|
| lipid bilayer after | 0.56 | 6.5 | | |
| vesicle fusion | | | | |
| lipid bilayer + | 0.60 | | 4.6 | 2818 |
| valinomycin | | | | |

The kinetics of the vesicle fusion by SPR is shown in FIG. 5 and the SPR and IS spectra before and after vesicle fusion are shown in FIGS. 6 and 7.

After incorporation of valinomycin (in the presence of potassium ions present in the buffer solution) the resistance of the layer decreases by 3 orders of magnitude. Moreover, the capacitance of the anchor region of 4.6 μF cm⁻² is firstly sufficiently different from the capacitance of the lipid layer (this results in a clear inflection point in the IS spectrum) and secondly they are very comparable to the anchored lipid layers described in the literature by Cornell et al (supra) that were prepared in a much more complicated manner.

1. Compounds comprising
(a) a carrier containing at least two functionalities and bound thereto:
(b) at least one saturated or unsaturated hydrocarbon or acyl residue with a chain length of 10-22 C atoms which can optionally be substituted by one or more side groups and
(c) a residue comprising a group (CH₂CH₂O)ₙ in which n=1-100 and a tethering group.

2. Compounds as claimed in claim 1,
characterized in that
the carrier contains three or four functionalities.

3. Compounds as claimed in claim 1 or 2,
characterized in that
the functionalities are selected from amino or/and hydroxyl groups.

4. Compounds as claimed in one of the claims 1 to 3,
characterized in that
the residues (b) and (c) are bound to the carrier by means of ether, thioether, ester or/and amide groups.

5. Compounds as claimed in claim 4,
characterized in that
the residues are bound by ether groups.

6. Compounds as claimed in one of the claims 1 to 5,
characterized in that
the carrier is selected from glycerol, 1-amino-2,3-propanediol, 2-amino-2-hydroxymethyl-1,3-propanediol.

7. Compounds as claimed in claim 6,
characterized in that
the carrier is glycerol.

8. Compounds of the general formulae (Ia) or (Ib) 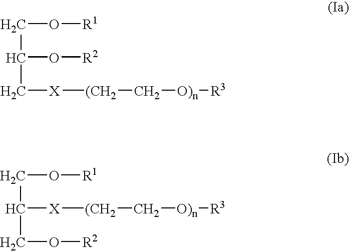
in which at least one of R¹ and R² independently denotes a saturated or unsaturated hydrocarbon residue or an acyl residue having a chain length of 10-22 C atoms which can optionally be substituted by one or several side groups, and the other is hydrogen, a C₁-C₉ hydrocarbon residue or a residue comprising a phospholipid, carboxyl, carbonyl, SO, SO₂, amide, amino or thiol group with or without a C₁-C₉ hydrocarbon residue,
n is an integer from 1-100,
R³ is a residue containing a tethering group and
X is a linking group.

9. Compounds as claimed in claim 8,
characterized in that
at least one of R¹ and R² is selected from saturated and unsaturated hydrocarbon residues.

10. Compounds as claimed in claim 9,
characterized in that
at least one of R¹ and R² is selected from saturated hydrocarbon residues which are substituted by one or more methyl groups.

11. Compounds as claimed in one of the claims 8 to 11,
characterized in that
X represents O.

12. Compounds as claimed in one of the claims 1 to 11,
characterized in that
n is an integer from 2-70.

13. Compounds as claimed in one of the claims 1 to 12,
characterized in that
the tethering group is selected from S-containing groups, phosphine groups and CN groups.

14. Compounds as claimed in claim 13,
characterized in that
the tethering group is an S—S group.

15. Compounds as claimed in claim 14,
characterized in that
the tethering group is a cyclic S—S group.

16. Compounds as claimed in one of the claims 1 to 15,
characterized in that
the tethering group represents a lipoic acid residue.

17. Compounds as claimed in one of the claims 1 to 16,
characterized in that
they are selected from 2,3-di-O-phytanyl-sn-glycero-1-tetraethyleneglycol-DL-α-lipoic acid ester, 2,3-di-O-phytanoyl-sn-glycero-1-tetraethyleneglycol-DL-α-lipoic acid ester, corresponding 1,2- or 1,3-diphytanyl or diphytanoyl derivatives and optical isomers thereof.

18. Supported biomimetic membrane comprising a lipid layer containing at least one compound of the general formula (I) as claimed in one of the claims 1 to 17 as a tethering lipid for anchoring to a substrate.

19. Supported membrane as claimed in claim 18,
characterized in that
the substrate has an electrically conductive surface.

20. Supported membrane as claimed in claim 19,
characterized in that
the substrate has a noble metal surface.

21. Supported membrane as claimed in claim 20,
characterized in that
the substrate has a gold surface.

22. Supported membrane as claimed in one of the claims 18 to 21,
characterized in that
the substrate has an ultra-smooth surface.

23. Supported membrane as claimed in one of the claims 18 to 22,
characterized in that
spacer molecules are additionally bound to the substrate.

24. Supported membrane as claimed in one of the claims 18 to 23 additionally comprising at least one mobile lipid.

25. Supported membrane as claimed in claim 24,
characterized in that
the mobile lipid is a phospholipid or a lipid that does not contain phosphorus provided with identical or different substituted or non-substituted alkyl chains with 8-22 carbon atoms which are linked to glycerol via ether or/and ester bonds, a glycolipid, a quartemary ammonium compound containing two C₁-C₄ alkyl groups and two C₈-C₁₈ alkyl chains, a tertiary amine containing two C₈-C₁₈ alkyl chains and a short C₁-C₄ alkyl chain or a hydrogen atom, a steroid or a chemically similar compound which is derived from the cholestane structure, or an isoprenoid as well as lipid mixtures comprising several of the said compounds wherein the lipids can optionally contain polar head groups such as hydroxyl, carboxyl, phosphoric acid esters and derivatives thereof, sulfur oxide derivatives with different oxidation stages of the sulfur, cholines, ethanolamines, inositols, carbohydrates, glycerols, aminoglycerols (in sphingosines) and amino acids and neutral, cationic or anionic forms or derivatives thereof.

26. Supported membrane as claimed in claim 25,
characterized in that
the mobile lipid of the lipid layer is selected from 1,2-di-O-phytanyl-sn-glycerophosphocholine, 1,2-di-O-phytanoyl-sn-glycerophosphocholine, 1,2-di-O-phytanyl-sn-glycerophosphoethanolamine, 1,2-di-O-phytanoyl-sn-glycerophosphoethanolaminc as well as corresponding 1,2- or 2,3-diphytanyl or diphytanoyl derivatives and mixtures of several of the said compounds.

27. Supported membrane as claimed in claim 26,
characterized in that
the lipid layer contains 1,2-di-O-phytanoyl-sn-glycero-phosphocholine as the mobile lipid.

28. Supported membrane as claimed in one of the claims 22 to 27,
characterized in that
it has a capacitance of 0.4 to 0.7 μF/cm².

29. Supported membrane as claimed in one of the claims 22 to 28,
characterized in that
it has a resistance of at least 1 MΩ·cm².

30. Supported membrane as claimed in one of the claims 18 to 29,
characterized in that
functional molecules are incorporated into the lipid layer.

31. Supported membrane as claimed in claim 30,
characterized in that
the functional molecules are selected from proteins, antibiotics, signal recognition molecules, messenger substances, porines, ion channels, membrane-changing substances such as narcotics, carriers, ligands, receptors, peptides, glycolipids and heterocyclic compounds.

32. Use of a supported membrane as claimed in one of the claims 18 to 31 as a biosensor.

33. Use as claimed in claim 32 in a detection method.

34. Use as claimed in claim 32 in a screening method.

35. Use as claimed in claim 34 to identify or/and characterize pharmacologically active substances.

36. Use as claimed in claim 32 to analyse biomolecules.

37. Use as claimed in claim 36 for the analysis of membrane-associated biomolecules selected from proteins, antibiotics, signal recognition molecules, messenger substances, porines, ion channels, membrane-changing substances such as narcotics, carriers, ligands, receptors, peptides, glycolipids and heterocyclic compounds.

38. Use as claimed in claim 32 as electrical insulation materials.
